(19) United States
(12) Patent Application Publication
(10) Pub. No.: US 2023/0109591 A1
(43) Pub. Date: Apr. 6, 2023

(54) DELIVERY CATHETER SYSTEMS

(71) Applicant: ReFlow Medical, Inc., San Clemente, CA (US)

(72) Inventors: Teodoro S. Jimenez, Jr., Aliso Viejo, CA (US); John Fulkerson, Rancho Santa Margarita, CA (US); Isa Rizk, San Diego, CA (US); Francisco Aguayo, Hemet, CA (US)

(21) Appl. No.: 17/956,672

(22) Filed: Sep. 29, 2022

Related U.S. Application Data
(60) Provisional application No. 63/251,213, filed on Oct. 1, 2021.

(51) Int. Cl. A61F 2/966; A61F 2/958

(52) U.S. Cl. CPC A61F 2/966; A61F 2/958

(57) ABSTRACT

Delivery systems for expandable elements, such as stents or scaffolds are described along with associated methods for using such systems. The delivery systems can be provided with a stent and/or a balloon the is enclosed by a sheath that need not extend an entire length of the delivery system. Instead, the sheath can be coupled to a control wire for retraction and advancement of the sheath. By enclosing the stent and/or balloon with a short sheath, the profile of the delivery system is reduced.

### CROSS-REFERENCE TO RELATED APPLICATIONS

This application claims the benefit of U.S. Provisional Application No. 63/251,213, entitled “Delivery Systems,” filed on Oct. 1, 2021, the disclosure of which is hereby incorporated herein by reference in its entirety.

### TECHNICAL FIELD

The present description relates generally to delivery systems for expandable elements, such as stents or scaffolds.

### BACKGROUND

A variety of devices can be used to deliver drugs or provide other therapies at desired treatment locations within a patient. For example, a stent, such as a drug-eluting stent (DES), can be positioned at the location of a stenosis (arterial narrowing) caused by arteriosclerosis. DESs generally include a drug containing polymer coated over a metal stent or scaffold, or a bioresorbable stent or scaffold composed of a drug-containing polymer. After a DES is delivered to a treatment location within a body lumen (e.g., vessel), it is expanded against a wall of the body lumen (e.g., a vessel wall) and the drug is released via direct contact with the wall. Direct delivery of the drug to the vessel wall enables significantly lower doses than those required via other delivery means (e.g., pills or injections).

### BRIEF DESCRIPTION OF THE DRAWINGS

FIG. 1 shows a partially schematic side view of an example of a delivery system.

FIG. 2 shows a cross-sectional view of an example region of the delivery system of FIG. 1 taken along line 2-2.

FIG. 3 shows a cross-sectional view of an example region of the delivery system of FIG. 1 taken along line 3-3.

FIG. 4 shows an enlarged, partially schematic side view of a distal portion of the delivery system of FIG. 1 with an unconstrained stent.

FIG. 5 shows a partially schematic side view of an example of a delivery system in an unassembled configuration.

FIG. 6 shows a side view of an example of a delivery system in a first stage of deployment.

FIG. 7 shows a side view of the delivery system of FIG. 6 in a second stage of deployment.

FIG. 8 shows a side view of the delivery system of FIG. 6 in a third stage of deployment.

FIG. 9 shows a side view of the delivery system of FIG. 6 in a fourth stage of deployment.

FIG. 10 shows a side view of an example of a delivery system in a first stage of deployment.

FIG. 11 shows a side view of the delivery system of FIG. 10 in another stage of deployment.

FIG. 12 shows a side view of an example of a delivery system in a first stage of deployment.

FIG. 13 shows a side view of the delivery system of FIG. 12 in another stage of deployment.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

The following disclosure describes various embodiments of delivery systems for expandable structures, such as stents or scaffolds, having spikes, flails, or other protruding features for penetrating target tissue and/or delivering drugs within a human patient, and associated devices and methods. The delivery systems can be configured to deliver and position expandable structures within a body lumen (e.g., vessel). In addition, these delivery systems can also be configured to deploy and expand the expandable structures in the body lumen. The delivery systems can further be configured to engage with the expanded structure and collapse the structure for removal from the body lumen. In some embodiments, the delivery systems can be configured to deliver another expandable structure or the same expandable structure to another body lumen, or the same body lumen, in a single procedure or during a plurality of procedures. Such delivery systems are expected to simplify and expedite transluminal procedures to more effectively deliver and position expandable structures within target tissues. The delivery systems can be used with more than one procedure, such as deployment of an expandable structure, when configured to re-capture the deployed expandable structure.

In particular, delivery systems described herein can be provided with a stent that is positioned over an inflatable balloon for expansion and delivery of the stent to a target delivery location. By positioning the stent over and about the inflatable balloon, the stent is ready to be expanded by the balloon immediately upon unsheathing with respect to the sheath. Additionally or alternatively, a stent can be positioned in an axially offset arrangement with respect to a balloon to reduce the need for space required by overlapping components.

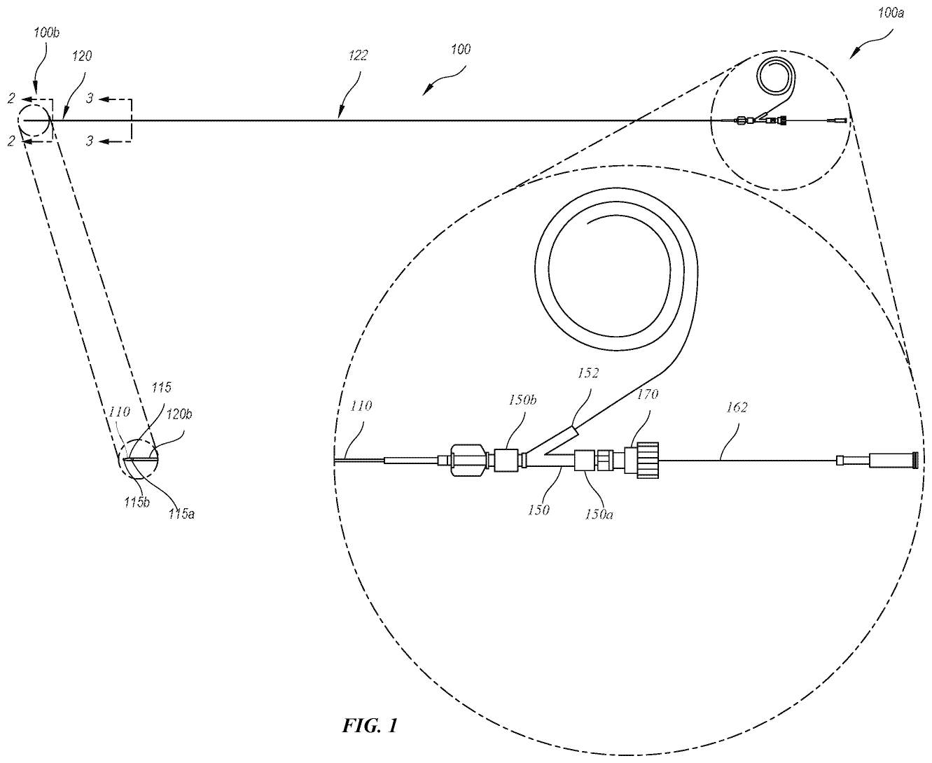

Certain details are set forth in the following description and FIGS. 1-13 to provide a thorough understanding of various embodiments of the disclosure. To avoid unnecessarily obscuring the description of the various embodiments of the disclosure, other details describing well-known structures and systems often associated with expandable structures, protruding features, and the components or devices associated with the manufacture of such structures are not set forth below. Moreover, many of the details and features shown in the figures are merely illustrative of particular embodiments of the disclosure. Accordingly, some embodiments can have other details and features without departing from the spirit and scope of the present disclosure. A person of ordinary skill in the relevant art will therefore understand that the present technology, which includes associated devices, systems, and procedures, may include other embodiments with additional elements or steps, and/or may include other embodiments without several of the features or steps shown and described below with reference to FIGS. 1-13. Furthermore, various embodiments of the disclosure can include structures other than those illustrated in the figures and are expressly not limited to the structures shown in the figures.

FIG. 1 shows a partially schematic side view of a delivery system 100 for a stent in a delivery state (e.g., low-profile or collapsed configuration). The delivery system 100 includes a sheath 120 (e.g., a tube) having a lumen for containing a distal portion of an inner shaft 110 and/or a guidewire 162. Proximal to the sheath 120, a sheath control wire 122 extends alongside (e.g., without enclosing) the inner shaft 110 and/or the guidewire 162. In some embodiments, the sheath 120 may also include one or more layers. In these embodiments, for example, the layers of the sheath 120 can include an inner layer, an outer layer, a liner, or a combination thereof. Each of the layers can be formed from materials including a polymer, high-density polyethylene (HDPE), polytetrafluoroethylene, silicone, Pebax® (polyether block amide) or a combination thereof. In some embodiments, each of the layers of the sheath 120 are formed from the same material. In other embodiments, however, one or more of the layers may be formed from different materials.

The inner shaft 110 can extend from a connector 150, through the sheath 120, and beyond the distal portion 120b of the sheath 120. The inner shaft 110 can be formed as a tubular structure (with or without a slit), such as a coiled tube, a braided tube, a reinforced tube, or a combination thereof, and may be constructed of a polymer material, such as a polyimide. The delivery system 100 can include a guidewire within the inner shaft 110 and accessible at a proximal end of the delivery system 100.

In the detailed view of the distal portion 100b of the delivery system 100, a tip 115 (e.g., an atraumatic tip) is disposed on a distal terminal end of the inner shaft 110. As illustrated, the tip 115 is adjacent to a distal terminal end of the sheath 120. At least a portion of the tip 115 can have the same cross-sectional dimension as the sheath 120, or the tip 115 may have a different cross-sectional dimension. In some embodiments, a distal end 115b of the tip 115 is tapered such that the distal end 115b has a smaller cross-sectional dimension compared to a proximal end 115a of the tip. Distal and/or proximal edges of the tip 115 may be curved/rounded so as to prevent the tip 115 from getting caught (e.g., stuck) on other portions of the delivery system 100 during delivery, positioning, deployment, etc. The tip 115 can be formed of the same material(s) as the sheath 120. In other embodiments, however, the tip 115 can be formed from different material(s) than the sheath 120.

The inner shaft 110 can be sized and shaped for intravascularly accessing a target site (e.g., treatment site) of the patient. In some embodiments, for example, the inner shaft 110 has a length of about 150 cm to about 180 cm and a suitable cross-sectional dimension for positioning within a subject's vasculature. The length of the inner shaft 110 can be a working length, such as a length that can be positioned within a subject's vasculature. In some embodiments, for example, the working length is about 70 cm to about 300 cm, about 150 cm to about 250 cm, or about 70 cm, about 80 cm, about 90 cm, about 100 cm, about 110 cm, about 120 cm, about 130 cm, about 140 cm, about 150 cm, about 160 cm, about 170 cm, about 180 cm, about 190 cm, about 200 cm, about 210 cm, about 220 cm, about 230 cm, about 240 cm, about 250 cm, about 260 cm, about 270 cm, about 280 cm, about 290 cm, or about 300 cm. In some embodiments, the sheath 120 has a length of about 1 centimeter (cm) to about 50 cm and a cross-sectional dimension of about 4 French, about 5 French, or about 6 French. In some embodiments, for example, the length of the sheath 120 is about 1 cm to about 50 cm, about 2 cm to about 40 cm, about 3 cm, about 4 cm, about 5 cm, about 6 cm, about 7 cm, about 8 cm, about 9 cm, about 10 cm, about 20 cm, about 30 cm, about 40 cm, and/or any value or range including or between the above values. In the illustrated embodiment, a sheath control wire 122 is coupled to the sheath 120 (e.g., via bonding and/or integrally or monolithically formed).

The delivery system 100 is configured to carry a stent, discussed further herein, in a delivery/collapsed state within a distal portion of the sheath 120. The stent can be at least partially covered by the sheath 120. In some embodiments, the stent can be fixedly or removably coupled to the inner shaft 110. Although the delivery system 100 is illustrated as a delivery system for stents, it will be appreciated that embodiments of the present technology can also include cages, meshes, balloons, membranes, tubular structures, circumferential bodies, expandable elements, expandable membranes, expandable structures, expandable tubular structures, and circumferentially expandable catheter tips with and without guidewire lumens.

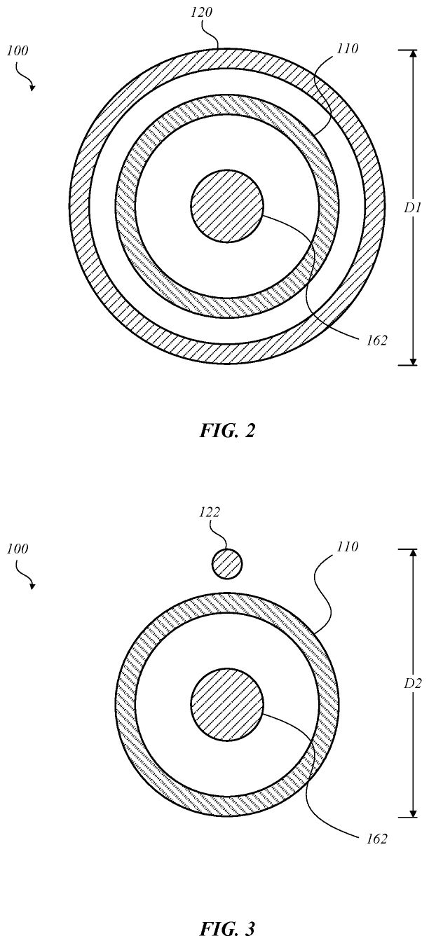

FIG. 2 shows a cross-sectional view of a region of the delivery system 100 of FIG. 1 taken along line 2-2. As illustrated in FIG. 2, the inner shaft 110 can be at least partially disposed within a lumen of the sheath 120 and the guidewire 162 can be at least partially disposed within a lumen of the inner shaft 110. In some embodiments, the sheath 120, the inner shaft 110, and/or the guidewire 162 each have a circular cross-sectional shape. In some embodiments, however, the sheath 120, the inner shaft 110, and/or the guidewire 162 can have other cross-sectional shapes, such as an ovoid shape, a “C” shape, a rectangular shape, a triangular shape, or the like.

The guidewire 162 and the inner shaft 110 can be positioned within the lumen of the sheath 120 in any configuration, such as anteriorly and posteriorly as illustrated, or medially and laterally. Furthermore, the guidewire 162 and the inner shaft 110 can be positioned in the lumen of the sheath 120 with respect to one another as illustrated, or the guidewire 162 can be positioned outside the inner shaft 110. A fluid pathway can be defined within the lumen of the inner shaft 110, for example along the length of the guidewire 162. The fluid pathway can connect to and/or be accessible by the port 152 of the connector 150.

FIG. 3 shows a cross-sectional view of a region of the delivery system 100 of FIG. 1 taken along line 3-3. As illustrated in FIG. 3, the inner shaft 110 can extend alongside the sheath control wire 122 and the guidewire 162 can be at least partially disposed within a lumen of the inner shaft 110. In some embodiments, the sheath control wire 122, the inner shaft 110, and/or the guidewire 162 each have a circular cross-sectional shape. In some embodiments, however, the sheath control wire 122, the inner shaft 110, and/or the guidewire 162 can have other cross-sectional shapes, such as an ovoid shape, a “C” shape, a rectangular shape, a triangular shape, or the like. The guidewire 162 and the inner shaft 110 can be positioned alongside the sheath control wire 122 without being enclosed thereby. The overall cross-sectional dimension D2 of the portion shown in FIG. 3 (i.e., with the sheath control wire 122 and without the sheath 120) can be less than the cross-sectional dimension D1 of the portion shown in FIG. 2 (i.e., with the sheath 120).

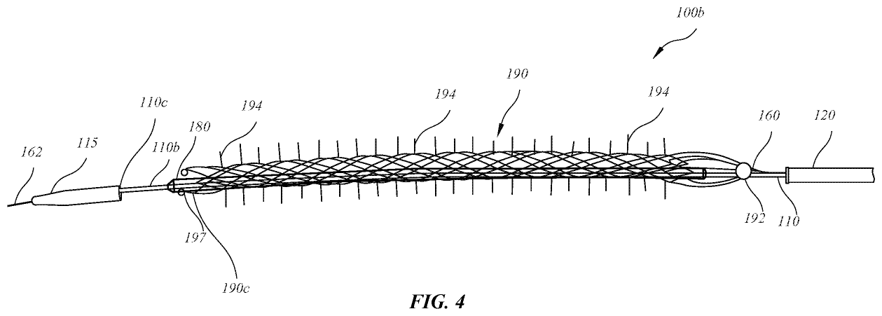

FIG. 4 shows a side view of a distal portion 100b of the delivery system 100 of FIG. 1 in a deployed state. In the illustrated embodiment, a stent 190 extends over a balloon 180 and is coupled to the inner shaft 110 and has been unsheathed from the sheath 120. A proximal visualization marker 192 is disposed on the stabilizing wire 160 near a proximal portion of the stent 190 and distal visualization markers 197 are disposed on a distal end 190c of the stent. In some embodiments, the proximal visualization marker 192 and/or the distal visualization marker 197 may be disposed on the stabilizing wire 160. The visualization markers 192 and/or 197 can be formed from any material that can be visualized while the stent 190 is intravascularly positioned (e.g., within a target blood vessel). In one embodiment, for example, the visualization markers 192 and/or 197 are radiopaque markers. The stabilizing wire 160 can be connected to the inner shaft 110, such that movement of the inner shaft 110 correspondingly urges the stent 190 via the stabilizing wire 160, as discussed further herein. Alternatively, the stabilizing wire 160 can be independently movable relative to the inner shaft 110, as discussed further herein.

The tip 115 is disposed on a terminal end 110c of the inner shaft 110 and can surround the terminal end 110c extending proximally along the distal portion 110b and/or distally from the terminal end 110c. The inner shaft 110 extends distally from the sheath 120, through a lumen of the stent 190, and, optionally, extends distally from the distal end of the stent 190. In the deployed configuration, protruding features 194 extend radially from a longitudinal axis of the stent 190, as discussed further herein.

The inner shaft 110 can also include an inflatable balloon (not shown), as discussed further herein. The inflatable balloon can be axially overlapping with the stent 190, distal to the stent 190, or proximal to the stent 190 while the stent 190 is in a delivery state (e.g., low-profile or collapsed configuration) within the sheath 120 and/or while the stent 190 is initially deployed from the delivery state.

The guidewire 162 can extend through the inner shaft 110 and beyond the tip 115. Accordingly, the guidewire 162 can be advanced ahead of other portions of the delivery system 100. The inner shaft 110, the stent 190, and the sheath 120 can be advanced over the guidewire 162 until the stent 190 is aligned with a desired target delivery location. The length of the guidewire 162 that overlaps other portions of the delivery system 100 can be within the inner shaft 110, so that it does not interfere with any other components of the delivery system 100.

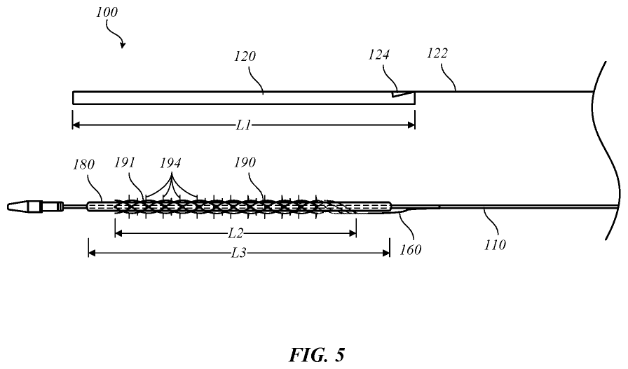

Referring now to FIG. 5, a sheath can enclose a portion of a delivery system while leaving a substantial length of the delivery system otherwise uncovered, while maintaining a connection for control of the sheath.

As shown in FIG. 5, the delivery system 100 is provided in an unassembled configuration, in which the sheath 120 is separate from the remaining components of the delivery system 100. As shown, the sheath 120 can extend along a length L1 that is greater than a length L2 of the stent 190 and/or a length L3 of the inflatable balloon 180 (e.g., whether overlapping or axially adjacent to each other). As such, the sheath 120 can cover and/or enclose the balloon 180 and/or the stent 190 in at least one configuration thereof. Such enclosure can include providing the sheath 120 along an entire axial length of the balloon 180 and/or the stent 190 and/or providing the sheath 120 along an entire circumferential distance about the balloon 180 and/or the stent 190. The length of the sheath 120 can be no more than 100%, 125%, 150%, 175%, 200%, 225%, 250%, 275%, or 300% of a length of the balloon 180 and/or the stent 190. The length of the sheath 120 can be no more than 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, or 20 cm longer than a length of the balloon 180 and/or the stent 190. As used herein, the length of the stent 190 refers to the length of a frame 191 that supports the protruding features 194. Such a length of the stent 190 excludes a length of the stabilization wire 160. For example, the length of the stent 190 can be measured from a distalmost end of the stent 190 to a proximalmost end that connects to the stabilization wire 160. As used herein, the length of the balloon 180 refers to the length thereof can controllably increase its outermost dimension and/or the length that extends radially beyond the inner shaft 110. The length of the balloon 180 can be measured from a distalmost end of the balloon 180 to a proximalmost end of the balloon 180. Such a length of the balloon 180 excludes lengths of the inner shaft 110 that extend proximally or distally beyond the balloon 180.

As further shown in FIG. 5, the sheath 120 can form a generally tubular and/or cylindrical shape to surround other components and maintain an otherwise low-profile. The sheath 120 can be longitudinally and/or circumferentially continuous, such that no radial openings are provided therethrough. In some embodiments, the outer surface and/or the inner surface of the sheath 120 can be smooth (e.g., circular), as further shown in FIG. 2. In some embodiments, the outer surface and/or the inner surface of the sheath 120 can be textured and/or form grooves, channels, recesses, cavities, and the like.

At a proximal end of the sheath 120, the sheath control wire 122 can be coupled thereto. For example, the sheath control wire 122 can be coupled to the sheath 120 by a connector 124. In some examples, the connector 124 transitions from a cross-sectional dimension of the sheath control wire 122 to a cross-sectional dimension of the sheath 120. While the sheath 120 can provide an inner lumen of its tubular shape to house the balloon 180 and/or the stent 190, the sheath control wire 122 can extend alongside other components of the delivery system 100 without enclosing such components. For example, the sheath control wire 122 can extend from a radial side (e.g., top, bottom, left, or right) of the sheath 120 at an axially terminal end thereof. This can leave the inner lumen of the sheath 120 unobstructed for passage of elements such as inner shaft 110. By further example, the sheath control wire 122 can extend alongside (e.g., generally in parallel with) the inner shaft 110.

By providing a sheath control wire 122 instead of a continuous sheath 120, the overall cross-sectional dimension of the delivery system 100 along the length traversed by the sheath control wire 122 can be smaller than it would be if the sheath 120 extended across such a length and enclosed the components along that length.

The sheath 120 can include one of a variety of structures to enclose the balloon 180 and/or the stent 190. For example, the sheath 120 can include a tube of a metal (e.g., stainless steel, titanium, nickel alloy, and the like), polymer (e.g., PEEK, HDPE, polyimide, and the like), and/or other material(s). In some embodiments, the sheath 120 can be a continuous structure that extends axially and/or circumferentially. In some embodiments, the sheath 120 can optionally include slits, gaps, openings, scoring, and/or other interruptions to provide greater flexibility along a length thereof. In some embodiments, the sheath 120 can be monolithic structure. In some embodiments, the sheath 120 can be formed as an assembly, such as a braided tube of helically extending filaments and/or a helical coil. In some embodiments, the sheath 120 can include one or more coating layers on inner and/or outer surfaces thereof. For example, such coatings can include a polymer (e.g., PTFE, polyether block amide, and the like) to facilitate movement of the sheath 120 while in contact with other components.

In some embodiments, the sheath 120 can be of a monolithic material, such as a metal, rather than an assembly of parts. As used herein, a monolithic structure is one that is integrally formed of a single piece of material, rather than of separate pieces that are joined together by an interface. By providing a monolithic, unitary, and/or unibody body, the sheath 120 does not contain interfaces or discontinuities therein, such as those that occur in assembled parts. Accordingly, the monolithic, unitary, and/or unibody body can be fabricated to more precise and consistent dimensions and provide smooth surfaces for interacting with the stent 190 and/or balloon 180 therein.

The sheath control wire 122 can include one of a variety of structures to facilitate control of the sheath 120. In some embodiments, the sheath control wire 122 can be a monolithic wire. In some embodiments, the sheath control wire 122 can be formed as an assembly, such as a braided wire of multiple filaments. For example, the sheath control wire 122 can be of a metal (e.g., stainless steel, titanium, nickel alloy, and the like). In some embodiments, the sheath 120 can include one or more coating layers on the outer surface thereof. For example, such coatings can include a polymer (e.g., PTFE, polyether block amide, and the like) and/or be joined with a coating of the sheath 120 to form a continuous coating. The sheath control wire 122 can provide adequate pushability and column strength to advance the sheath 120 as well as retract it with tension. In some embodiments, multiple sheath control wires 122 are provided extending proximally from the sheath 120.

Methods described herein provide delivery of the stent 190 to a target delivery location by operation of the delivery system 100. While methods in their various stages are discussed and illustrated herein, it will be understood that multiple variations of each method are also contemplated. For example, the methods can be performed in various orders of operations, with additional operations, or with fewer operations.

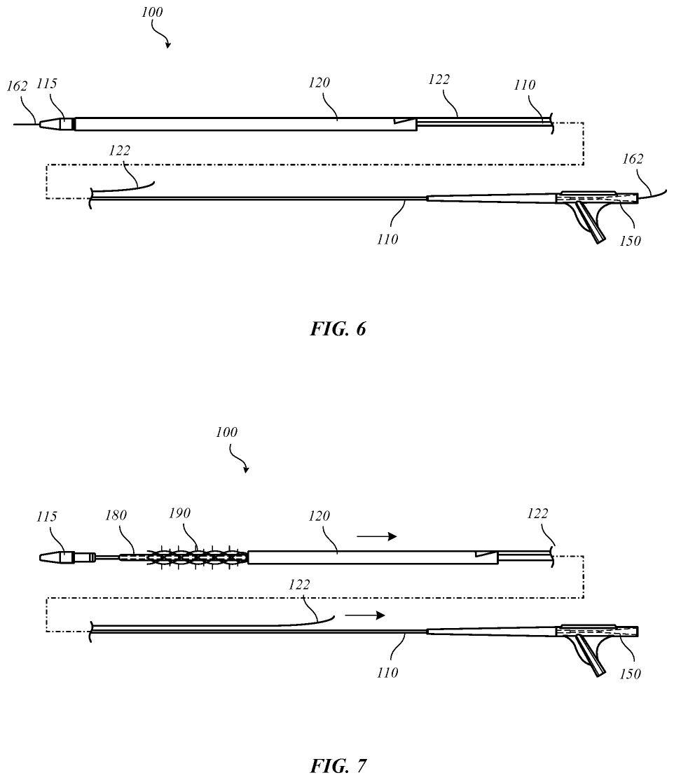

As shown in FIGS. 6-9, a delivery system 100 can be provided with a stent 190 that is positioned over an inflatable balloon 180 for expansion and delivery of the stent 190 to a target delivery location. By positioning the stent 190 over and about the inflatable balloon 180, the stent 190 is ready to be expanded by the balloon 180 immediately upon unsheathing with respect to the sheath 120.

As shown in FIG. 6, the delivery system 100 is provided with the sheath 120 covering other components of the delivery system 100. For example, the sheath 120 can extend to the tip 115 positioned at a distal end of the inner shaft 110. The inner shaft 110 can extend to and into the sheath 120, with a length thereof accessible proximal to the sheath 120 (e.g., alongside the sheath control wire 122). Additionally or alternatively, the connector 150 can be accessible proximal to the sheath 120 (e.g., alongside the sheath control wire 122). As discussed above, a guidewire 162 can be advanced ahead of the tip 115 (e.g., through the inner shaft 110) to provide a pathway for advancement of other components of the delivery system 100. A proximal end of the guidewire 162 can be accessed through the inner shaft 110, for example through a sidewall opening therein.

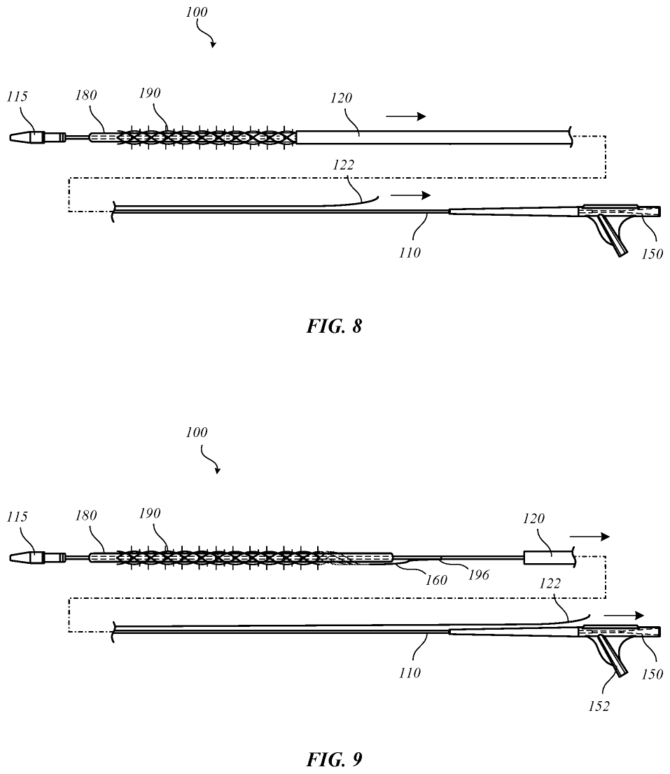

As shown in FIGS. 7 and 8, the sheath 120 can be moved to unsheathe the stent 190 and other components of the delivery system 100. For example, once the distal region of the delivery system 100 is positioned at a desired location, the sheath 120 is configured to be at least partially proximally retracted relative to the inner shaft 110 by retracting the sheath control wire 122 relative to the connector 150. Once the sheath 120 is partially retracted, at least a portion of the stent 190 and/or the balloon 180 is unsheathed and protruding features 194 of the stent 190 are configured to radially expand outwardly away from the inner shaft 110.

As used herein, movement of various components can be relative to other components of the delivery system 100 and/or relative to a position apart from the delivery system 100 (e.g., a position within the anatomy of the patient, target delivery location, and/or tissue). The directions “proximal” and “distal” can be with respect to the delivery system 100, a component thereof, and/or a position apart from the delivery system 100. For example, movement of the guidewire 162 can be relative to the sheath 120, the inner shaft 110, the stent 190, and/or the balloon 180. It will be understood that while the guidewire 162 moves, the sheath 120, the inner shaft 110, the stent 190, and/or the balloon 180 can be stationary, moving in the same direction (e.g., at a different speed), or moving in a different (e.g., opposite) direction. It will be further understood that while the sheath 120, the inner shaft 110, the stent 190, and/or the balloon 180 moves, the guidewire 162 can be stationary, moving in the same direction (e.g., at a different speed), or moving in a different (e.g., opposite) direction. By further example, movement of the sheath 120 can be relative to the inner shaft 110, the stent 190, and/or the balloon 180. It will be understood that while the sheath 120 moves, the inner shaft 110, the stent 190, and/or the balloon 180 can be stationary, moving in the same direction (e.g., at a different speed), or moving in a different (e.g., opposite) direction. By further example, movement of the inner shaft 110, the stent 190, and/or the balloon 180 can be relative to sheath 120. It will be understood that while the inner shaft 110, the stent 190, and/or the balloon 180 move, the sheath 120 can be stationary, moving in the same direction (e.g., at a different speed), or moving in a different (e.g., opposite) direction.

As shown in FIG. 9, the sheath 120 has been retracted and the stent 190 is unsheathed. The stabilizing wire 160, connected to the inner shaft 110 by the anchor portion 196, is configured to engage with the proximal end of the stent 190 and control the position of the stent 190 during and after retraction of the sheath 120. Accordingly, the position of the stent 190 is maintained with respect to the inner shaft 110, including the balloon 180. For example, while some adjustment of the length and/or axial position of the stent 190 may occur during radial expansion of the stent 190, it will be understood that the stabilizing wire 160 can maintain the position of at least a portion of the stent 190 to be around and axially aligned with at least a portion of the balloon 180. The balloon 180 can have an axial length that is greater than the axial length of the stent 190, so that an entirety of the stent 190 is overlapping with the balloon 180. As shown in FIG. 9, the stabilizing wire 160 can connect the stent 190 to a portion of the inner shaft 110 that is proximal to the balloon 180. Additionally or alternatively, the stabilizing wire 160 can connect the stent 190 to a portion of the inner shaft 110 that is distal to the balloon 180.

When both the stent 190 and the balloon 180 are unsheathed by the sheath 120 and exposed, the balloon 180 can be inflated to expand or further expand the stent 190. For example, an interior region of the balloon can be fluidly connected, via the inner shaft 110, to the port 152 of the connector 150. By providing a fluid through the port 152, the balloon 180 can be expanded, thereby expanding or further expanding the stent 190. The expansion with respect to target anatomy will be further discussed herein.

Following one or more of the above-described operations, the balloon 180 can be deflated. The stent 190 can be maintained for any duration of time in an expanded state. For example, the stent 190 can be maintained for a duration of time effective to provide therapeutic treatment (e.g., remodeling and/or drug delivery) to target anatomy and allows fluid flow through the expanded stent and deflated balloon where there is no fluid blockage through the treated site.

Additionally or alternatively, the delivery system 100 can be deployed at multiple locations. The stent 190 can be collapsed by moving the sheath 120 over the stent 190. The stent 190 and the balloon 180 can be moved to another target location, and one or more of the above-described operations can be repeated.

Additionally or alternatively, the delivery system 100 can be removed. The stent 190 can be collapsed by moving the sheath 120 over the stent 190. Components of the delivery system 100 can be removed from the patient by retracting proximally over the guidewire.

Additionally or alternatively, the stent 190 can be detached from the inner shaft 110 and left as an implant within the patient. Following detachment, other components of the delivery system 100 can be removed from the patient by retracting proximally over the guidewire.

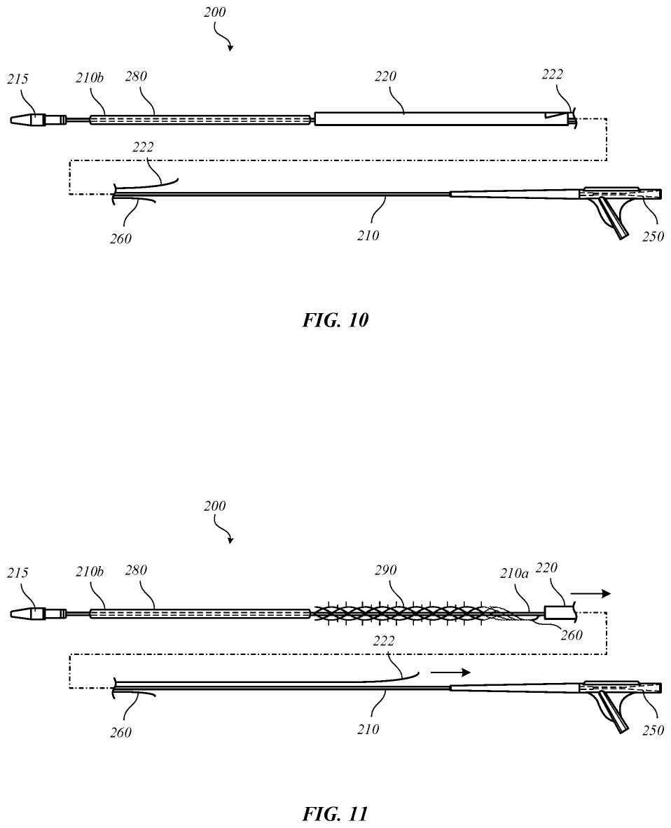

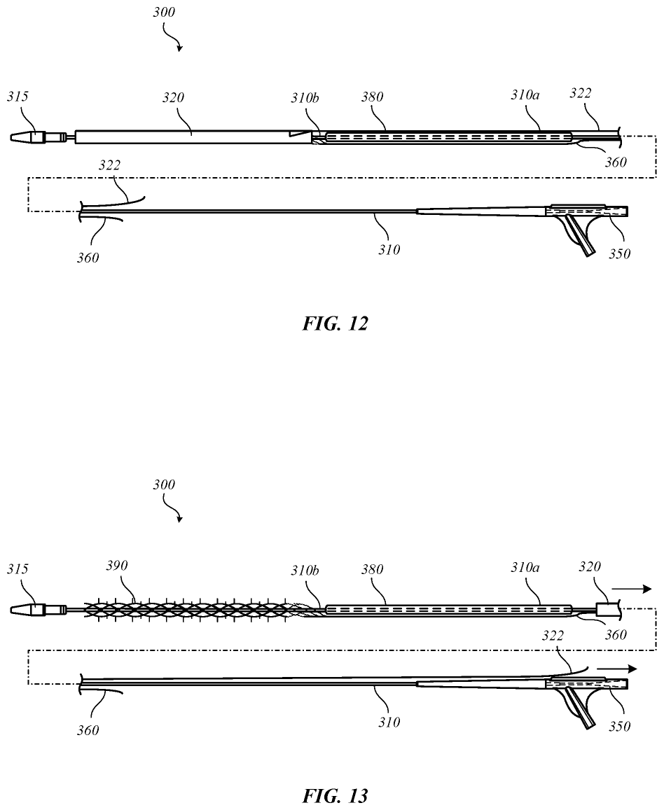

While the delivery system 100 is shown with a stent 190 positioned over a balloon 180 in a delivery state, it will be understood that other arrangements are contemplated. For example, a stent can be positioned in an axially offset arrangement with respect to a balloon to reduce the need for space required by overlapping components. Reference is made to a delivery system 200, as shown in FIGS. 10 and 11 and a delivery system 300, as shown in FIGS. 12 and 13. While each of the delivery system 200 and the delivery system 300 is in some aspects different than the delivery system 100, it will be understood that components and features of the delivery system 100 as described herein can apply to either or both of the delivery system 200 and the delivery system 300. Similar or like items can perform the same function as those shown in the delivery system 100, and the features of such items are not all discussed hereafter, for brevity.

As shown in FIGS. 10 and 11, the delivery system 200 can be provided with a stent 290 that is positioned proximal to an inflatable balloon 280 for expansion and delivery of the stent 290 to a target delivery location. By positioning the stent 290 proximal to the inflatable balloon 280, the stent 290 and the balloon 280 are not overlapping (e.g., are axially offset) while in a delivery state within the sheath 220, and thereby reduce the space requirements within the sheath 220.

As further shown in FIGS. 10 and 11, the sheath 220 can be moved to unsheathe the stent 290. For example, once the distal region of the delivery system 200 is positioned at a desired location, the sheath 220 is configured to be at least partially proximally retracted relative to the inner shaft 210 by retracting the sheath control wire 222 relative to the connector 250. Once the sheath 220 is partially retracted, at least a portion of the balloon 280 and at least a portion of the stent 290 are unsheathed. Protruding features 294 of the stent 290 are configured to radially expand outwardly away from the inner shaft 210. As shown, the balloon 280 is positioned at a distal portion 210b of the inner shaft 210, and the stent 290 is positioned at a proximal portion 210a of the inner shaft 210. The proximal portion 210a of the inner shaft 210 can have an outer cross-sectional dimension that is smaller than an outer cross-sectional dimension of the balloon 280, thereby permitting the stent 290 to be collapsed onto the proximal portion 210a in a smaller profile than would be achieved if the stent 290 were collapsed onto the balloon 280.

The stabilizing wire 260, accessible by a user, is configured engage with the proximal end of the stent 290 and control the position of the stent 290 during and after retraction of the sheath 220. The user can secure the stabilizing wire 260 relative to the inner shaft 210 while the sheath 220 is retracted so that the position of the stent 290 can be maintained with respect to the inner shaft 210, including the balloon 280, during retraction of the sheath 220.

When both the stent 290 and the balloon 280 are unsheathed by the sheath 220 and exposed, the stent 290 can be axially aligned with the balloon 280. Because the inner shaft 210 extends through the stent 290, proximal retraction of the inner shaft 210 relative to the stent 290 can achieve axially alignment of the balloon 280 with the stent 290. The balloon 280 can have an axial length that is greater than the axial length of the stent 290, so that an entirety of the stent 290 is overlapping with the balloon 280 when axially aligned. Additionally or alternatively, the inner shaft 210 and the sheath 220 can be retracted together with respect to the stent 290.

As shown in FIGS. 12 and 13, the delivery system 300 can be provided with a stent 390 that is positioned distal to an inflatable balloon 380 for expansion and delivery of the stent 390 to a target delivery location. By positioning the stent 390 distal to the inflatable balloon 380, the stent 390 and the balloon 380 are not overlapping (e.g., are axially offset) while in a delivery state within the sheath 320, and thereby reduce the space requirements within the sheath 320.

As further shown in FIGS. 12 and 13, the sheath 320 can be moved to unsheathe the stent 390. For example, once the distal region of the delivery system 300 is positioned at a desired location, the sheath 320 is configured to be at least partially proximally retracted relative to the inner shaft 310 by retracting the sheath control wire 322 relative to the connector 350. Once the sheath 320 is partially retracted, a portion of the stent 390 is unsheathed and protruding features 394 of the stent 390 are configured to radially expand outwardly away from the inner shaft 310. The stabilizing wire 360, accessible by a user, is configured engage with the proximal end of the stent 390 and control the position of the stent 390 during and after retraction of the sheath 320. The user can secure the stabilizing wire 360 relative to the inner shaft 310 while the sheath 320 is retracted so that the position of the stent 390 can be maintained with respect to the inner shaft 310, including the balloon 380, during retraction of the sheath 320.

When both the stent 390 and the balloon 380 are unsheathed by the sheath 320 and exposed, the stent 390 can be axially aligned with the balloon 380. Because the inner shaft 310 extends through the stent 390, distal movement of the inner shaft 310 relative to the stent 390 can achieve axially alignment of the balloon 380 with the stent 390. The balloon 380 can have an axial length that is greater than the axial length of the stent 390, so that an entirety of the stent 390 is overlapping with the balloon 380 when axially aligned.

While the stents described herein have the features shown, it will be understood that a variety of different stents and other devices can be used with the delivery systems described herein. Various features are set forth below by way of example, and not by limitation.

Regarding such stents and other devices, the material(s) for forming the frame, struts, and/or protruding features described herein can be selected based on mechanical and/or thermal properties, such as strength, ductility, hardness, elasticity, flexibility, flexural modulus, flexural strength, plasticity, stiffness, emissivity, thermal conductivity, specific heat, thermal diffusivity, thermal expansion, any of a variety of other properties, or a combination thereof. If formed from a material having thermal properties, the material can be activated to deliver thermal treatment to the desired treatment site. Regardless of the material, the frame, struts, and/or protruding features can be formed from a tube or a wire, such as a solid wire, by laser cutting or other suitable techniques. When formed from the wire, a portion of the wire can be removed by chemical etching or another suitable method to create an inner dimension of the stent.

The embodiments described herein provide delivery systems for one or more structures having a means for delivering drugs to a specific region within a body lumen, such as the vasculature, while still allowing fluid (e.g., blood) to flow through the treatment area where the structure has been placed and/or other devices or treatment means within the adjacent body lumen. In some embodiments, the fluid is temporary prevented from flowing through the treatment area while one or more regions of systems is delivered, deployed, positioned, and/or removed from the body lumen. In addition, the delivery systems can be configured to prepare the body lumen for treatment, by raking the stent, pulling the stent, turning the stent, or a combination thereof, proximal or distal to the treatment site. In some embodiments, the delivery systems can be configured to rotate the stent when mechanical force is applied.

The systems disclosed herein can provide for adjustment, recapture, and/or redeployment of the associated stents or other structures, and/or deployment of a different stent or other structure, allowing a practitioner to more effectively to treat a desired region more accurately and deliberately. In several embodiments, the stent or other delivery structure can be deployed for a temporary period (e.g., for less than 24 hours), and then retracted and removed. In these embodiments, the protruding features can engage with and/or pierce the lumen wall and remain therein after the stent or other delivery structure is removed, or can be retracted and removed with the stent or other delivery structure. The stent can be configured to self-expand, or partially self-expand, when deployed from the delivery system and also be configured to further expand within the body lumen when the balloon is expanded therein. The stent can also be configured to post-dilate when removed from the body lumen. In some embodiments, the stent or other delivery structure can be deployed for a long-term temporary period (e.g., for less than 2 weeks, less than one month, less than 6 months, less than one year), and then retracted and removed. In some embodiments, a different stent or delivery structure can be deployed after a first stent or delivery structure has been retraced and removed. The duration of deployment and duration after removal before deployment of the different stent or delivery structure can vary from minutes, to hours, to days, to weeks, to months, or to years. In these embodiments, removal of the first stent or delivery structure and deployment of a different stent or delivery structure can occur once, twice, three times, four times, five times, six times, seven times, eight times, nine times, or ten times. Moreover, the embodiments described herein can allow for a lower profile system than currently available systems.

In the embodiments described herein and other embodiments configured in accordance with the present technology, stents and other expandable structures may include non-protruding features, such as deployable and/or expandable features, that are not configured for delivering a drug to a target location. For example, stents and other expandable structures configured in accordance with the present technology can include one or more protruding features, one or more non-protruding features, or combinations thereof.

While many embodiments of the stents and/or structures described herein include stents, additional embodiments of the expandable elements, such as stents and/or structures, can include non drug-eluting stents and/or non drug-eluting structures. In these embodiments, the non drug-eluting stents may include one or more protruding members, such as spikes. The spikes can be configured to engage with and/or penetrate a portion of the body lumen or vessel. For example, the spikes can penetrate the vessel wall, thereby reducing and/or eliminating an elasticity of the vessel wall. In these embodiments, the protruding members can be configured to prevent the vessel wall from progressing inward toward the body lumen and restricting and/or constricting flow therein. The protruding members can be integrally formed with the struts, or disposed on the surface of the struts, extending radially outward from the struts toward the target tissue.

Various examples of aspects of the disclosure are described below as clauses for convenience. These are provided as examples, and do not limit the subject technology.

Clause A: a delivery system comprising: an inner shaft comprising an inflatable balloon; a stent disposed around the balloon and coupled to the inner shaft; a sheath extending circumferentially continuously around the stent and the inflatable balloon; and a sheath control wire coupled to a proximal end of the sheath and extending proximally from the sheath alongside the inner shaft.

Clause B: a delivery system comprising: an inner shaft comprising an inflatable balloon; a stent extending about a portion of the inner shaft; a sheath covering an entirety of the stent, wherein the sheath has a length that is less than 200% of a length of the stent; and a sheath control wire coupled to a proximal end of the sheath and extending alongside the inner shaft.

Clause C: a delivery system comprising: an inner shaft comprising an inflatable balloon; a stent extending about a portion of the inner shaft, the stent being axially adjacent to the balloon; a sheath covering the stent without covering the balloon; and a sheath control wire coupled to a proximal end of the sheath and extending proximally from the sheath alongside the inner shaft.

One or more of the above clauses can include one or more of the features described below. It is noted that any of the following clauses may be combined in any combination with each other, and placed into a respective independent clause, e.g., clause A, B, or C.

Clause 1: the stent comprises a frame and protruding features configured to extend radially away from the frame when the stent is released from the sheath.

Clause 2: the sheath has a length that is less than 200% of a length of the stent.

Clause 3: the sheath has a length that is no more than 10 cm greater than a length of the stent.

Clause 4: the delivery system has a cross-sectional dimension at the sheath control wire that is less than a cross-sectional dimension at the sheath.

Clause 5: a guidewire slidably disposed within the inner shaft.

Clause 6: the sheath further covers an entirety of the balloon.

Clause 7: the length of the sheath is less than 200% of a length of the balloon.

Clause 8: the length of the sheath is no more than 10 cm greater than a length of the balloon.

Clause 9: the stent and the sheath are proximal to the balloon.

Clause 10: the stent and the sheath are distal to the balloon.

A reference to an element in the singular is not intended to mean one and only one unless specifically so stated, but rather one or more. For example, “a” module may refer to one or more modules. An element proceeded by “a,” “an,” “the,” or “said” does not, without further constraints, preclude the existence of additional same elements.

Headings and subheadings, if any, are used for convenience only and do not limit the invention. The word exemplary is used to mean serving as an example or illustration. To the extent that the term include, have, or the like is used, such term is intended to be inclusive in a manner similar to the term comprise as comprise is interpreted when employed as a transitional word in a claim. Relational terms such as first and second and the like may be used to distinguish one entity or action from another without necessarily requiring or implying any actual such relationship or order between such entities or actions.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A phrase “at least one of” preceding a series of items, with the terms “and” or “or” to separate any of the items, modifies the list as a whole, rather than each member of the list. The phrase “at least one of” does not require selection of at least one item; rather, the phrase allows a meaning that includes at least one of any one of the items, and/or at least one of any combination of the items, and/or at least one of each of the items. By way of example, each of the phrases “at least one of A, B, and C” or “at least one of A, B, or C” refers to only A, only B, or only C; any combination of A, B, and C; and/or at least one of each of A, B, and C.

It is understood that the specific order or hierarchy of steps, operations, or processes disclosed is an illustration of exemplary approaches. Unless explicitly stated otherwise, it is understood that the specific order or hierarchy of steps, operations, or processes may be performed in different order. Some of the steps, operations, or processes may be performed simultaneously. The accompanying method claims, if any, present elements of the various steps, operations or processes in a sample order, and are not meant to be limited to the specific order or hierarchy presented. These may be performed in serial, linearly, in parallel or in different order. It should be understood that the described instructions, operations, and systems can generally be integrated together in a single software/hardware product or packaged into multiple software/hardware products.

In one aspect, a term coupled or the like may refer to being directly coupled. In another aspect, a term coupled or the like may refer to being indirectly coupled.

Terms such as top, bottom, front, rear, side, horizontal, vertical, and the like refer to an arbitrary frame of reference, rather than to the ordinary gravitational frame of reference. Thus, such a term may extend upwardly, downwardly, diagonally, or horizontally in a gravitational frame of reference.

The disclosure is provided to enable any person skilled in the art to practice the various aspects described herein. In some instances, well-known structures and components are shown in block diagram form in order to avoid obscuring the concepts of the subject technology. The disclosure provides various examples of the subject technology, and the subject technology is not limited to these examples. Various modifications to these aspects will be readily apparent to those skilled in the art, and the principles described herein may be applied to other aspects.

All structural and functional equivalents to the elements of the various aspects described throughout the disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for”.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirements of the applicable patent law, nor should they be interpreted in such a way.

What is claimed is:

1. A delivery system comprising:
an inner shaft comprising an inflatable balloon;
a stent disposed around the balloon and coupled to the inner shaft;
a sheath extending circumferentially continuously around the stent and the inflatable balloon; and
a sheath control wire coupled to a proximal end of the sheath and extending proximally from the sheath alongside the inner shaft.

2. The delivery system of claim 1, wherein the stent comprises a frame and protruding features configured to extend radially away from the frame when the stent is released from the sheath.

3. The delivery system of claim 1, wherein the sheath has a length that is less than 200% of a length of the stent.

4. The delivery system of claim 1, wherein the sheath has a length that is no more than 10 cm greater than a length of the stent.

5. The delivery system of claim 1, wherein the delivery system has a cross-sectional dimension at the sheath control wire that is less than a cross-sectional dimension at the sheath.

6. The delivery system of claim 1, further comprising a guidewire slidably disposed within the inner shaft.

7. A delivery system comprising:
an inner shaft comprising an inflatable balloon;
a stent extending about a portion of the inner shaft;
a sheath covering an entirety of the stent, wherein the sheath has a length that is less than 200% of a length of the stent; and
a sheath control wire coupled to a proximal end of the sheath and extending alongside the inner shaft.

8. The delivery system of claim 7, wherein the length of the sheath is no more than 10 cm greater than the length of the stent.

9. The delivery system of claim 7, wherein the sheath further covers an entirety of the balloon.

10. The delivery system of claim 9, wherein the length of the sheath is less than 200% of a length of the balloon.

11. The delivery system of claim 9, wherein the length of the sheath is no more than 10 cm greater than a length of the balloon.

12. The delivery system of claim 7, wherein the stent comprises a frame and protruding features configured to extend radially away from the frame when the stent is released from the sheath.

13. The delivery system of claim 7, wherein the delivery system has a cross-sectional dimension at the sheath control wire that is less than a cross-sectional dimension at the sheath.

14. The delivery system of claim 7, further comprising a guidewire slidably disposed within the inner shaft.

15. A delivery system comprising:
an inner shaft comprising an inflatable balloon;
a stent extending about a portion of the inner shaft, the stent being axially adjacent to the balloon;
a sheath covering the stent without covering the balloon; and
a sheath control wire coupled to a proximal end of the sheath and extending proximally from the sheath alongside the inner shaft.

16. The delivery system of claim 15, wherein the stent and the sheath are proximal to the balloon.

17. The delivery system of claim 15, wherein the stent and the sheath are distal to the balloon.

18. The delivery system of claim 15, wherein the stent comprises a frame and protruding features configured to extend radially away from the frame when the stent is released from the sheath.

19. The delivery system of claim 15, wherein the delivery system has a cross-sectional dimension at the sheath control wire that is less than a cross-sectional dimension at the sheath.

20. The delivery system of claim 15, further comprising a guidewire slidably disposed within the inner shaft.
